(19) United States
(12) Patent Application Publication
(10) Pub. No.: US 2016/0175601 A1
(43) Pub. Date: Jun. 23, 2016

(54) SYSTEMS AND METHODS FOR MANAGING TIERED TACHYCARDIA THERAPY

(71) Applicant: PACESETTER, INC., Sylmar, CA (US)

(72) Inventors: Yelena Nabutovsky, Mountain View, CA (US); Gene A. Bornzin, Simi Valley, CA (US); John W. Poore, South Pasadena, CA (US); Edward Karst, Los Angeles, CA (US)

(21) Appl. No.: 14/575,894

(22) Filed: Dec. 18, 2014

(51) Int. Cl. A61N 1/38; A61N 1/365; A61N 1/375; A61N 1/362; A61N 1/39

(52) U.S. Cl. CPC A61N 1/385; A61N 1/3621; A61N 1/3956; A61N 1/3756; A61N 1/365

(57) ABSTRACT

Systems and methods are provided for managing tiered tachycardia therapy. The systems and methods measure a cardiac feature of interest (FOI) from a cardiac electrical signal of a heart sensed from at least one electrode of a leadless cardiac pacemaker (LPM). The systems and methods detect when the cardiac FOI satisfies an arrhythmia criteria, and deliver anti-tachycardia pacing (ATP) to the heart using the at least one electrode of the LPM when the cardiac FOI satisfies the arrhythmia criteria. The systems and methods deliver a series of arrhythmia emulating (AE) pulses configured to emulate a cardiac arrhythmia to the heart using the at least one electrode of the LPM if the FOI in cardiac signals measured subsequent to the delivery of the ATP therapy satisfy the arrhythmia criteria.

### BACKGROUND

Embodiments of the present disclosure generally relate to administering tiered tachycardia therapy, and, more particularly, for administering the tiered therapy from a leadless pacemaker and a subcutaneous implantable cardioverter defibrillator.

A subcutaneous implantable cardioverter defibrillator (S-ICD) is generally a defibrillator that is implanted under the skin, for example, on a side of the chest of a patient below the arm pit. Similar to conventional intravenous implantable cardioverter defibrillators (I-ICD), the S-ICD provides electrical shocks to the heart for the treatment of abnormal heartbeats. The S-ICD has several benefits over I-ICDs such as reduced implant complications, easier implant procedure, less cosmetic impact, and no vein damage.

However, currently available S-ICDs have very limited pacing capabilities compared to traditional defibrillators, for example, S-ICDs are not able to deliver anti-tachycardia pacing (ATP). ATP therapy generally is the use of pacing stimulation techniques for termination of tachyarrhythmia, such as, terminating ventricular tachycardia (VT). ATP therapy is used to treat VT. For each episode of VT, if ATP is effective, a high voltage shock is avoided. If it is not effective, shock is delivered to terminate the VT. ATP therapy reduces the overall use of the ICD shock therapy, which has been shown to cause discomfort and some degree of psychological distress to the patient, reducing the patient's quality of life.

Leadless pacemakers (LPM) may be configured to administer ATP therapy, which used concurrently with the S-ICD, may be used to administer both ATP and shock therapy. To coordinate operations between the LPM and the S-ICD, communication messages are transmitted between the devices from electrodes or using radio frequency means. However, these communication messages require both devices to have dedicated communication modules and/or resources (e.g., memory) with protocol information to discern received communication messages, which are not present in commercially available S-ICD. There is a need for a method and/or system for commercially available S-ICD and the LPM to interact and coordinate with each other to effectively provide appropriate ICD shock and ATP therapies to the patient without requiring additional system components.

### SUMMARY

In at least one embodiment, a method is provided for managing tiered tachycardia therapy. The method includes measuring a cardiac feature of interest (FOI) from a cardiac electrical signal of a heart sensed from at least one electrode of a leadless cardiac pacemaker (LPM). The method includes detecting when the cardiac FOI satisfied an arrhythmia criteria, and delivering anti-tachycardia pacing (ATP) therapy to the heart using the at least one electrode of the LPM when the cardiac FOI satisfies the arrhythmia criteria. The method further includes delivering a series of arrhythmia emulating (AE) pulses configured to emulate a cardiac arrhythmia using the at least one electrode of the LPM if the cardiac FOI in cardiac signals measured subsequent to the delivery of the ATP therapy satisfy the arrhythmia criteria.

In at least one embodiment, a system is described for managing tiered tachycardia therapy. The system includes a leadless pacemaker (LPM). The LPM includes sensing circuitry within a housing of the LPM. The sensing circuitry is configured to sense a cardiac electrical signal of a heart through at least one electrode. The system also includes a controller circuit within the housing of the LPM. The controller circuit is configured to measure a cardiac feature of interest (FOI) from the cardiac electrical signal, detect when the cardiac FOI satisfies an arrhythmia criteria, and deliver anti-tachycardia pacing (ATP) therapy to the heart using the at least one electrode of the LPM when the cardiac FOI satisfies the arrhythmia criteria. The controller circuit is also configured to generate a series of arrhythmia emulating (AE) pulses configured to emulate a cardiac arrhythmia if the cardiac FOI in pulses measured subsequent to the delivery of ATP therapy satisfy the arrhythmia criteria.

### BRIEF DESCRIPTION OF THE DRAWINGS

FIG. 1 is a perspective view of a system for managing tiered tachycardia therapy, according to an embodiment of the present disclosure

FIG. 2 is a schematic block diagram of a leadless pacemaker, according to an embodiment of the present disclosure.

FIG. 3 is a flowchart of a method for managing tiered tachycardia therapy, according to an embodiment of the present disclosure.

FIG. 4 is a graphical representation of a cardiac electrical signal sensed by a leadless pacemaker, according to an embodiment of the present disclosure.

FIG. 5 is a line graph of cardiac features of interest based on the cardiac electrical signal from FIG. 4.

FIG. 6 is a graphical representation of a cardiac electrical signal sensed by a leadless pacemaker with a predetermined number of anti-tachycardia pacing bursts, according to an embodiment of the present disclosure.

FIG. 7 is a graphical representation of a cardiac electrical signal sensed by a leadless pacemaker with a predetermined number of anti-tachycardia pacing bursts, according to an embodiment of the present disclosure.

FIG. 8A is a graphical representation of a cardiac electrical signal sensed by a leadless pacemaker, according to an embodiment of the present disclosure.

FIG. 8B is graphical representation of a series of arrhythmia emulating pulses delivered by the leadless pacemaker of FIG. 8A.

FIG. 8C is a graphical representation of the cardiac electrical signal of FIG. 8A overlaid with the series of arrhythmia emulating pulses of FIG. 8B.

FIG. 9 is a schematic block diagram of a subcutaneous implantable cardiac defibrillator, according to an embodiment of the present disclosure.

FIG. 10 is a peripheral view of a leadless pacemaker, according to an embodiment of the present disclosure.

### DETAILED DESCRIPTION

Embodiments of the present disclosure provide systems and methods for delivering tiered tachycardia therapy using two or more implantable medical devices, for example, a leadless pacemaker (LPM) and a subcutaneous implantable cardioverter defibrillator (S-ICD) without intravenous leads. The tiered tachycardia therapy may correspond to different actions taken by the LPM and S-ICD based on a cardiac feature of interest (FOI), such as, a heart rate or cardiac signal morphology, from a cardiac electrical signal of a heart of the patient.

For example, the S-ICD may be programmed to deliver an implantable cardioverter defibrillator (ICD) shock therapy when a ventricular fibrillation (VF) is detected based on the cardiac FOI. The LPM may deliver an anti-tachycardia pacing (ATP) therapy when the cardiac FOI satisfies arrhythmia criteria, such as criteria corresponding to ventricular tachycardia (VT). If the cardiac FOI continues to satisfy the arrhythmia criteria after the ATP therapy is supplied by the LPM, the LPM may generate a series of arrhythmia emulating (AE) pulses to simulate an electrophysiologic pattern of VF, triggering the S-ICD to deliver ICD shock therapy.

At least one technical effect of various embodiments described herein include coordinating a pre-existing or implanted S-ICD to deliver ICD shock therapy, when the LPM delivers AE pulses once sensed by the S-ICD. At least one technical effect of various embodiments described herein include coordinating functions (e.g., ATP therapy, ICD shock therapy) of the LPM and the S-ICD without transmitting communication messages between the LPM and the S-ICD.

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

As used herein, the term “leadless” generally refers to an absence of electrically-conductive leads that traverse vessels, while “intra-venous” means generally with electrically-conductive leads that traverse vessels, such as the SVC, IVC, CS, pulmonary arteries and the like.

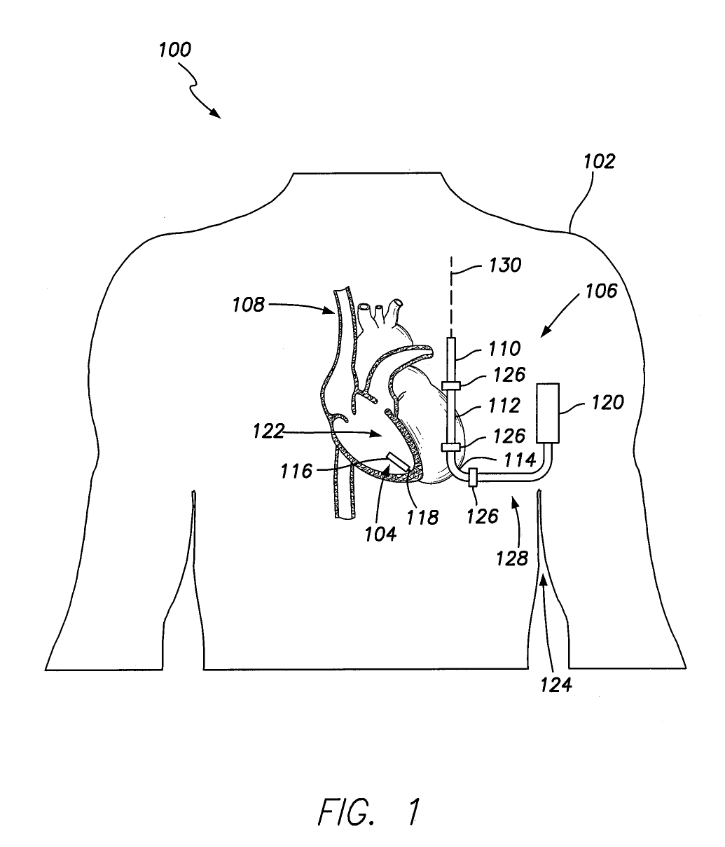

FIG. 1 is a perspective view of a heart 108 with an implantable medical system 100 for managing tiered tachycardia therapy, according to an embodiment of the present disclosure. The system 100 may include at least a leadless implantable medical device such as a leadless pacemaker (LPM) 104 and a subcutaneous implantable cardioverter defibrillator (S-ICD) 106. Optionally, the system 100 may include more than one LPM 104.

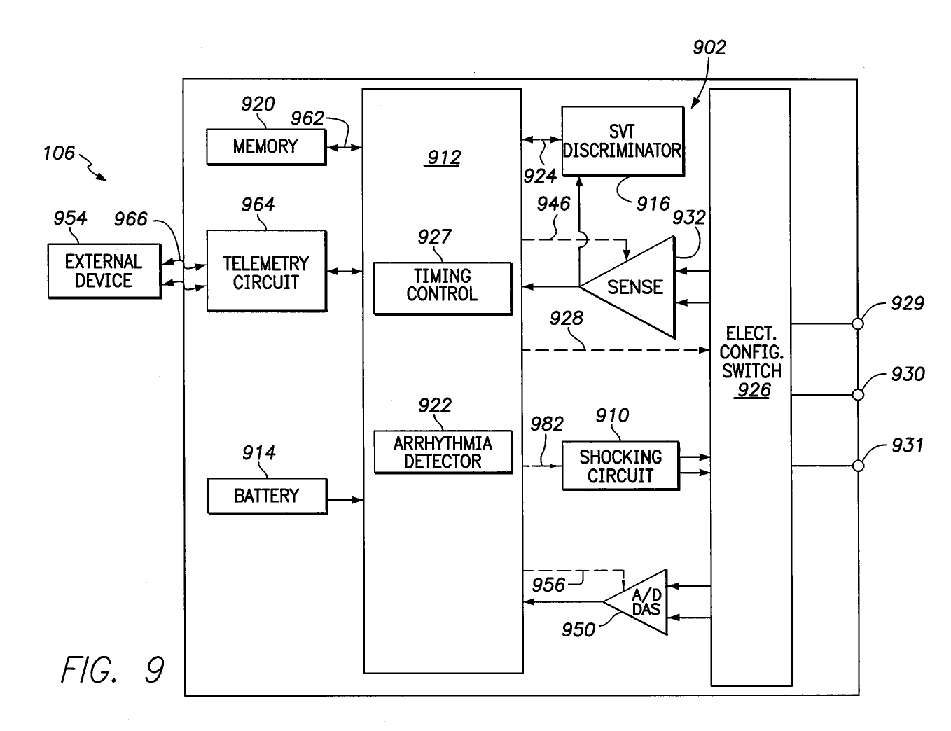

The S-ICD 106 includes a housing 120 implanted on a side of a chest of a patient 102 proximate to an arm pit 124. The housing 120 is coupled to a lead 128, which conducts an ICD shock therapy (e.g., shocking pulses) generated by a shocking circuit 910 (FIG. 9) from within the housing 120. The S-ICD 106 may be programmed to deliver the ICD shock therapy at a predetermined ICD shock threshold corresponding to at least one of ventricular fibrillation (VF) or ventricular tachycardia (VT) based on measurements of the cardiac FOI. The predetermined ICD shock threshold may be based on the ICD threshold testing.

The ICD shock therapy is applied to tissue of the patient 102 via lead electrodes 110-114. The lead electrodes 110-114 may be positioned proximate to the heart 108 along a vertical axis 130 of the lead 128, and are angularly positioned about the vertical axis 130 such that the lead electrodes 110-114 do not overlap. Optionally, the lead 128 may include a pre-shaped bend to allow one or more electrodes 110-114 to be positioned proximate to the LPM 104.

The lead electrodes 110-114 may be in the shape of a ring such that each lead electrode 110-114 continuously covers the circumference of the exterior surface of the lead 128. Each of the lead electrodes 110-114 are separated by non-conducting rings 126, which electrically isolate each lead electrodes 110-114 from an adjacent lead electrodes 110-114. The non-conducting rings 126 may include one or more insulative material and/or bio-compatible materials to allow the lead 128 to be implantable within the patient 102. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane.

One or more of the lead electrodes 110-114 may be configured to emit the ICD shock therapy in an outward radial direction proximate to the heart 108 and/or be configured to create a cardiac event sensing channel used for sensing electrical activity (e.g., cardiac electrical signals) from the heart 108, such as, the sub-Q surface ECG between the lead electrodes 110-114. For example, the lead electrode 112 may be configured to emit the ICD shock therapy and the lead electrodes 110 and 114 may be configured to sense electrical activity between the lead electrodes 110 and 114.

In the example of FIG. 1, the LPM 104 is implanted in a right ventricle (RV) 122 of the heart 108 to administer pacing pulses and sense heart beats within the RV 122. Additionally or alternatively, the LPM 104 and/or other LPMs may be implanted in the left ventricle (LV), the right atrium (RA), and/or the left atrium (LA). Optionally, the LPM 104 may be configured for dual-chamber functionality from a primary location within a single chamber of the heart (e.g., the RV 122). For example, the LPM 104 may include both atrial and ventricular sensing and pacing circuitry. Optionally, additional LPMs may be implanted in other chambers of the heart 108 with the LPM 104 to allow dual-chamber pacing, or three-chamber pacing without requiring pacing lead connections to the S-ICD 106.

The LPM 104 includes a hermetically sealed housing 116 with a proximal end 118 that is configured to engage local tissue of interest, such as, the right ventricle 122. The housing 116 is configured to be implanted entirely within a single local chamber of the heart 108 and to hold the electronic/computing components of the LPM 104. The internal electrical components and electrodes may be implemented as described in U.S. patent application Ser. No. 13/653,248, filed Oct. 16, 2012 (Docket A12P1044), and Ser. No. 13/866,803, filed Apr. 19, 2013, the complete subject matter of which are expressly incorporated herein by reference in its entirety.

For convenience, hereafter the chamber in which the LPM 104 is implanted shall be referred to as the “local” chamber. The local chamber includes a local chamber wall that physiologically responds to local activation events originating within the local chamber. The local chamber is at least partially surrounded by local wall tissue that forms or constitutes at least part of a conduction network for the associated chamber. For example, during normal operation, the wall tissue of the right atrium contracts in response to an intrinsic local activation event that originates at the sino-atrial (SA) node and in response to conduction that propagates along the atrial wall tissue. For example, tissue of the right atrium chamber wall in a healthy heart follows a conduction pattern, through depolarization, that originates at the SA node and moves downward about the right atrium until reaching the atrioventricular (AV) node. The conduction pattern moves along the chamber wall as the right atrium wall contracts.

The term “adjacent” chamber shall refer to any chamber separated from the local chamber by tissue (e.g., the RV and LA are adjacent chambers to the RA; the RA and LV are adjacent chambers to the LA; the RA and RV are adjacent to one another; the RV and LV are adjacent to one another, and the LV and LA are adjacent to one another).

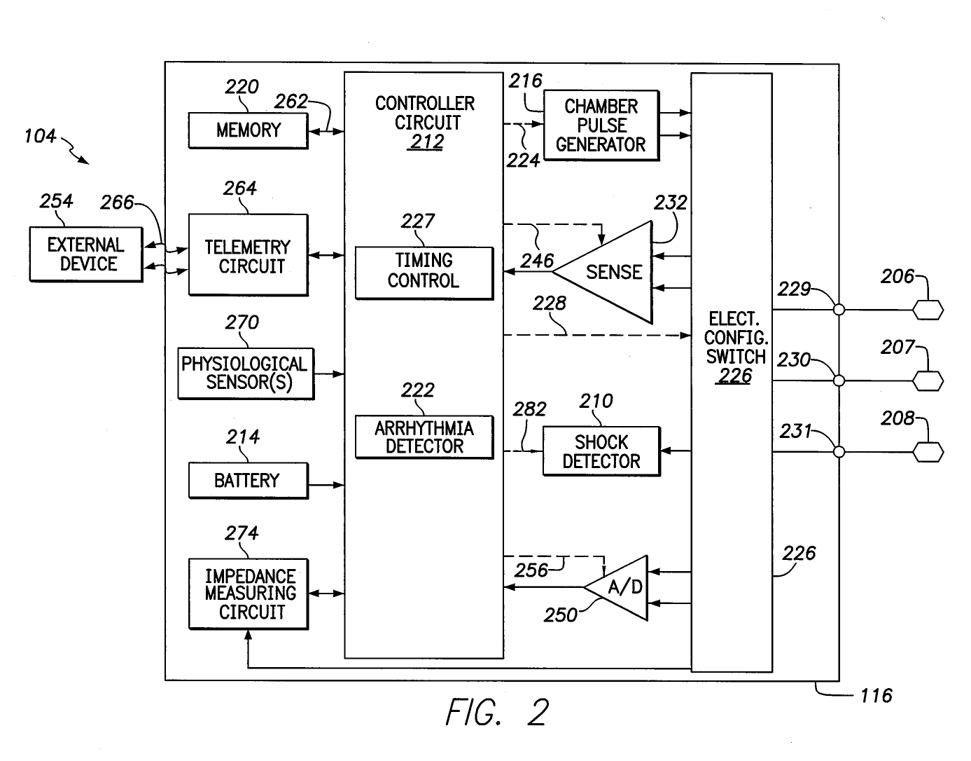

FIG. 2 is a schematic block diagram of the LPM 104 and shows the LPM's functional elements substantially enclosed in the housing 116. The housing 116 (which is often referred to as the “can”, “case”, “encasing”, or “case electrode”) may be programmably selected to act as the return electrode for certain stimulus modes. The LPM 104 is shown having three electrodes 206-208 located within, on, or near the housing 116, for delivering pacing pulses (e.g., anti-tachycardia pacing, anti-bradycardia pacing) to and sensing electrical activity from the muscle of the local chamber (e.g., the RV 122). The electrodes 206-208 may be the same size or at least two electrodes 206-208 have different sizes. The electrodes 206-208 engage the local chamber wall tissue at a tissue of interest for a local activation site that is near the surface of the wall tissue, and that is within the conduction network of the local chamber. The type and location of each electrode 206-208 may vary. For example, the electrodes 206-208 may include various combinations of ring electrodes, tip electrodes, coil electrodes and the like. It should be noted that in other embodiments the LPM 104 may have more than or less than three electrodes 206-208 (e.g., one) that what is illustrated in FIG. 2.

A plurality of terminals or Hermetic feedthroughs 229-231 conduct electrode signals through the housing 116 that interface with the electrodes 206-208 of the LPM 104. For example, the feedthroughs 229-231 may include: a feedthrough 229 that connects with a first electrode associated with the housing (e.g. the electrode 206) and located in the local chamber; a feedthrough 230 that connects with a second electrode associated with the housing (e.g., the electrode 207) and located in the local chamber; a feedthrough 231 that connects with a third electrode associated with the housing (e.g. the electrode 208) and located in the local chamber and possibly partially extending into tissue associated with an adjacent chamber. The housing 116 contains a battery 214 to supply power for pacing, sensing, and/or other functions of the LPM 104 described herein. The housing 116 also contains sensing circuitry 232 for sensing cardiac activity through the electrodes 206 and 208 and a pulse generator 216. The sensing circuitry 232 is configured to detect and/or sense electrical activity, such as physiologic and pathologic behavior and events sensed from the electrodes 206-208.

The electrodes 206-208 receive stimulus pulse(s) generated from the pulse generator 216 for delivery by one or more of the electrodes 206-208 coupled thereto. The pulse generator 216 is controlled by the controller circuit 212 via a control signal 224. The pulse generator 216 is coupled to the select electrode(s) via an electrode configuration switch 226, which includes multiple switches for connecting the desired electrodes to the appropriate I/O circuits, thereby facilitating electrode programmability. The switch 226 is controlled by a control signal 228 from the controller circuit 212.

In the example of FIG. 2, a single pulse generator 216 is illustrated. Optionally, the LPM 104 may include multiple pulse generators, similar to the pulse generator 216, where each pulse generator is coupled to one or more electrodes and controlled by the controller circuit 212 to deliver select stimulus pulse(s) to the corresponding one or more electrodes.

The controller circuit 212 is illustrated as including timing control circuitry 227 to control the timing of the stimulation pulses (e.g., pacing rate, atrio-ventricular (AV) delay, or the like). The timing control circuitry 227 may also be used for the timing of refractory periods, blanking intervals, noise detection windows, evoked response windows, alert intervals, marker channel timing, and the like. The controller circuit 212 also has an arrhythmia detector 222 for detecting arrhythmia conditions, for example, based on one or more cardiac FOI measured by the sensing circuitry 232 and/or the controller circuit 212. Although not shown, the controller circuit 212 may further include other dedicated circuitry and/or firmware/software components that assist in monitoring various conditions of the heart 108 and managing pacing therapies.

The sensing circuitry 232 is selectively coupled to one or more electrodes through the switch 226. The sensing circuitry 232 detects the presence of cardiac activity in the local chamber of the heart 108. In at least one embodiment, the sensing circuitry 232 may detect the presence of cardiac activity in adjacent chambers of the heart 108. The sensing circuitry 232 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the LPM 104 to sense low amplitude signal characteristics of atrial fibrillation. The switch 226 determines the sensing polarity of the cardiac signal by selectively closing the appropriate switches. In this way, the clinician may program the sensing polarity independent of the stimulation polarity.

The output of the sensing circuitry 232 is connected to the controller circuit 212 which, in turn, triggers or inhibits the pulse generator 216 in response to the absence or presence of cardiac activity. The sensing circuitry 232 receives a control signal 246 from the controller circuit 212 for purposes of controlling the gain, threshold, polarization charge removal circuitry (not shown), and the timing of any blocking circuitry (not shown) coupled to the inputs of the sensing circuitry.

In the example of FIG. 2, a single sensing circuit 232 is illustrated. Optionally, the LPM 104 may include multiple sensing circuits, similar to the sensing circuit 232, where each sensing circuit is coupled to one or more electrodes 206-208 and controlled by the controller circuit 212 to sense electrical activity detected at the corresponding one or more electrodes. The sensing circuit 232 may operate in a unipolar sensing configuration or in a bipolar sensing configuration.

The LPM 104 may further include an analog-to-digital (ND) data acquisition system (DAS) 250 coupled to one or more electrodes 206-208 via the switch 226 to sample cardiac signals across any pair of desired electrodes. The data acquisition system 250 may be configured to acquire intracardiac electrogram signals, convert the raw analog data into digital data, and store the digital data for later processing and/or telemetric transmission to an external device 254 (e.g., a programmer, local transceiver, or a diagnostic system analyzer). The data acquisition system 250 is controlled by a control signal 256 from the controller circuit 212.

The controller circuit 212 is coupled to the memory 220 by a suitable data/address bus 262. The programmable operating parameters used by the controller circuit 212 may be stored in memory 220 and used to customize the operation of the LPM 104 to suit the needs of the patient 102. Such operating parameters define, for example, pacing pulse amplitude, pulse duration, electrode polarity, rate, sensitivity, automatic features, arrhythmia detection criteria, and the amplitude, wave shape and vector of each shocking pulse to be delivered to the heart 108 within each respective tier of therapy.

The operating parameters of the LPM 104 may be non-invasively programmed into the memory 220 through a telemetry circuit 264 in telemetric communication via a communication link 266 with the external device 254. The telemetry circuit 264 allows intracardiac electrograms and status information relating to the operation of the LPM 104 (as contained in the controller circuit 212 or the memory 220) to be sent to the external device 254 through the established communication link 266. Additionally or alternatively, the LPM 104 may be equipped with a communication modem (modulator/demodulator) to enable wireless communication with a remote device, such as a second implanted LPM 104 in a master/slave arrangement, such as described in U.S. Pat. No. 7,630,767.

The LPM 104 may further include one or more physiologic sensors 270. Such sensors are commonly referred to as “rate-responsive” sensors because they are typically used to adjust pacing stimulation rates according to the exercise state of the patient. However, the physiological sensor 270 may further be used to detect changes in cardiac output, changes in the physiological condition of the heart, or diurnal changes in activity (e.g., detecting sleep and wake states). Signals generated by the physiological sensors 270 are passed to the controller circuit 212 for analysis. The controller circuit 212 responds by adjusting the various pacing parameters (such as rate, AV Delay, V-V Delay, etc.) at which the atrial and ventricular pacing pulses are administered. While shown as being included within the LPM 104, the physiologic sensor(s) 270 may be external to the LPM 104, yet still be implanted within or carried by the patient. Examples of physiologic sensors might include sensors that, for example, sense respiration rate, pH of blood, ventricular gradient, activity, position/posture, temperature, minute ventilation (MV), and so forth.

The battery 214 provides operating power to all of the components in the LPM 104. The battery 214 is capable of operating at low current drains for long periods of time, and is capable of providing high-current pulses. The battery 214 also desirably has a predictable discharge characteristic so that elective replacement time can be detected. As one example, the LPM 104 employs lithium/silver vanadium oxide batteries.

The LPM 104 may further include an impedance measuring circuit 274, which may be used for many things, including: impedance surveillance during the acute and chronic phases for proper LPM 104 positioning or dislodgement; detecting operable electrodes and automatically switching to an operable pair if dislodgement occurs; measuring respiration or minute ventilation; measuring thoracic impedance; detecting when the device has been implanted; measuring stroke volume; and detecting the opening of heart valves; and so forth. The impedance measuring circuit 274 is coupled to the switch 226 so that any desired electrode may be used.

Optionally, the LPM 104 may include a shock detector circuit 210. The shock detector circuit 210 may be used to detect electrical shocks corresponding to ICD shock therapy delivered by the S-ICD 106. For example, the shock detector circuit 210 may identify an artificial cardioversion or defibrillation shock pulse corresponding to ICD shock therapy based on sensed electric signals received from the sensing circuitry 232 through one or more electrodes 206-208. The shock detector circuit 210 may output a detection signal 982 to the controller circuit 212. In response to the detection signal the controller circuit 212 may instruct the pulse generator 216 to deliver post-shock pacing from one or more of the electrodes 206-208 of the LPM 104.

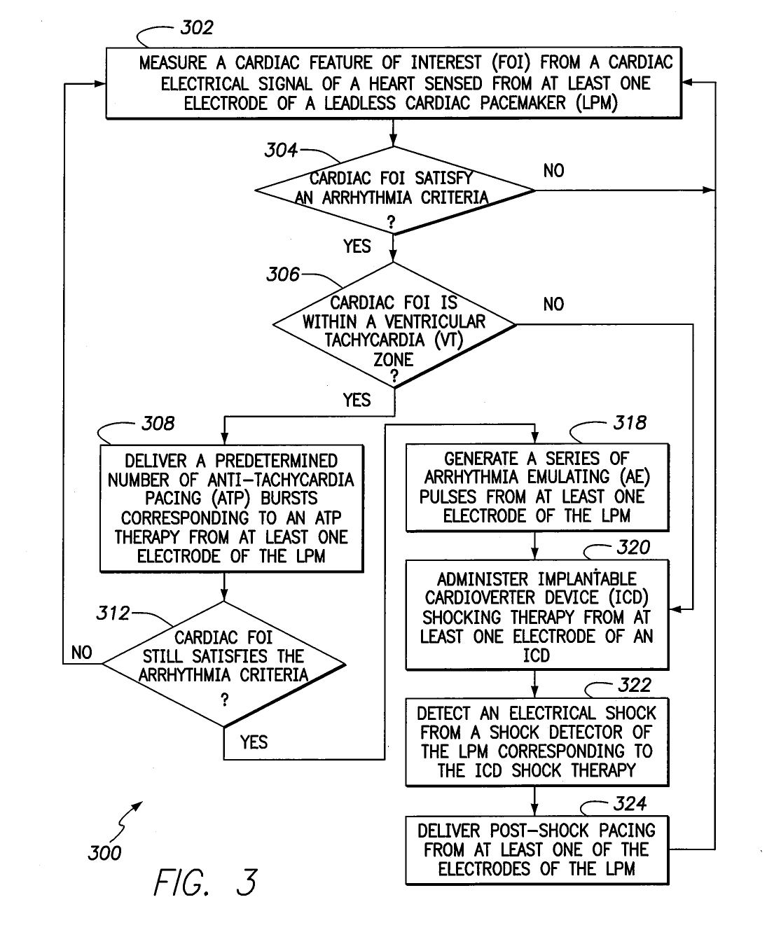

FIG. 3 is a flowchart of a method 300 for administering tiered tachycardia therapy. The method 300, for example, may employ structures or aspects of various embodiments (e.g., systems and/or methods) discussed herein. For example, the leadless cardiac pacemaker (LPM) may be similar to the LPM 104 (FIGS. 1 and 2) or may include other features, such as those described or referenced herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed or repeated in an iterative fashion. It should be noted, other methods may be used, in accordance with embodiments herein.

One or more methods may include (i) measuring a cardiac feature of interest (FOI) from a cardiac electrical signal of a heart sensed from at least one electrode of a leadless cardiac pacemaker (LPM), (ii) detecting when the cardiac FOI satisfies an arrhythmia criteria, (iii) delivering a predetermined number of anti-tachycardia pacing (ATP) bursts corresponding to an ATP therapy from the at least one electrode of the LPM when the cardiac FOI satisfies the arrhythmia criteria, and (v) generating a series of arrhythmia emulating (AE) pulses from the at least one electrode of the LPM if the FOI satisfies the arrhythmia criteria after the predetermined number of ATP bursts are delivered.

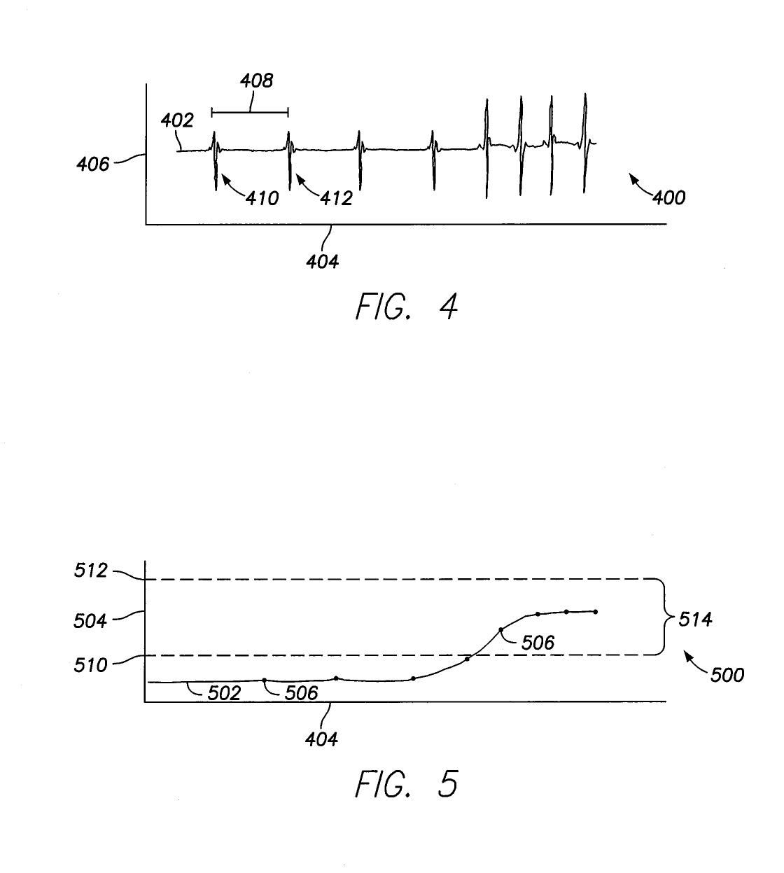

Beginning at 302, the method 300 measures a cardiac feature of interest (FOI) from a cardiac electrical signal 402 of the heart 108 sensed from at least one electrode (e.g., the electrodes 206-208) of the LPM 104. FIG. 4 is a graphical representation 400 of the cardiac electrical signal 402 sensed by the LPM 104. The cardiac electrical signal 402 is plotted over a horizontal axis 404 representing time and a vertical axis 406 representing current and/or voltage. The cardiac FOI may represent a heart rate of the heart 108. For example, the sensing circuitry 232, through the electrode 206, senses electrical activity of the cardiac electrical signal 402 of the heart 108. The controller circuit 212 receives the sensed cardiac electrical signal 402 from the sensing circuitry 232 and measures the cardiac FOI, which may be the heart rate, from the cardiac electrical signals 402 based on R-R intervals, such as the R-R interval 408, between adjacent QRS complexes, for example the QRS complexes 410-412, of the cardiac electrical signal 402. Optionally, the cardiac FOI may be an average heart rate based on more than one R-R interval between adjacent QRS complexes 410-412 of the sensed cardiac electrical signal 402 over a set number of cardiac cycles (e.g., number of QRS complexes).

At 304, the method 300 detects when the cardiac FOI 506 satisfies an arrhythmia criteria. FIG. 5 is an illustration 500 of a line graph 502 of measurements by the controller circuit 212 of the cardiac FOI 506, for example the heart rate, based on the cardiac electrical signal 402 sensed by the sensing circuit 232. The vertical axis 504 represents the heart rate, such as, beats per minute. In at least one embodiment, the controller circuit 212 may compare the cardiac FOI 506 with an arrhythmia threshold 510 to detect if the cardiac FOI 506 satisfies the arrhythmia criteria. In at least one embodiment, the arrhythmia criteria may be based on the heart rate (e.g., the cardiac FOI 506) of the heart, which is used to define the arrhythmia threshold 510.

For example, the arrhythmia criteria may correspond to instances when the heart rate is above 170 beats per minute. Based on the above arrhythmia criteria, the controller circuit 212 may be programmed or select the arrhythmia threshold 510 be defined at 170 beats per minutes. Based on the arrhythmia threshold 510, the controller circuit 212 may detect that cardiac FOI 506 satisfies the arrhythmia criteria when the cardiac FOI 506 is above the arrhythmia threshold 510. It should be noted that in other embodiments the arrhythmia threshold 510 may be greater than or less than 170 beats per minute. Optionally, the arrhythmia threshold 510 may be adjusted based on an average of the cardiac FOI 506 over time. Additionally or alternatively, the controller circuit 212 may compare the cardiac FOI 506, such as a QRS complex, measured from the sensed cardiac electrical signal 402 with a QRS complex morphology template stored in the memory 220 of a LPM to detect when the arrhythmia criteria is satisfied. Optionally, the arrhythmia criteria may correspond to the heart rate remaining above the arrhythmia threshold 510 for a predetermined time period. For example, the arrhythmia criteria may correspond to the heart rate being above 170 beats per minute for twelve intervals (e.g., R-R intervals).

At 306, the method 300 determines if the cardiac FOI is within a ventricular tachycardia (VT) zone 514. The VT zone 514 may represent a subset within the arrhythmia criteria that corresponds to cardiac FOI 506 that represent a VT of the heart 108. In at least one embodiment, the VT zone 514 may be a range of heart rates defined by the arrhythmia threshold 510 and a VF threshold 512.

For example, the VF threshold 512 may be set at a heart rate of 220 beats per minute. The arrhythmia threshold 510 is set by the controller circuit 212 at 170 beats per minute. The controller circuit 212, based on the VF threshold 512 and the arrhythmia threshold 510, may determine that sensed cardiac electrical signals 402 with heart rates between the thresholds 510, 512, or 170 and 220 beats per minute are within the VT zone 514. It should be noted that in other embodiments the VT zone 514 may have a range greater than or less than heart rates between 170 and 220, and/or include heart rates greater than or less than 170 and/or 220.

If the cardiac FOI 506 is within the VT zone 514, then at 308, the method 300 delivers a predetermined number of anti-tachycardia pacing (ATP) bursts 606, 706 corresponding to an ATP therapy from the at least one electrode of the LPM 104 when the cardiac FOI (e.g., 506) satisfies the arrhythmia criteria. The predetermined number of ATP bursts 606, 706 may be stored in the memory 220. The ATP therapy may include delivering one or more ATP bursts 606, with ramp pacing (e.g., ATP burst 706), or other known ATP therapies known in the art.

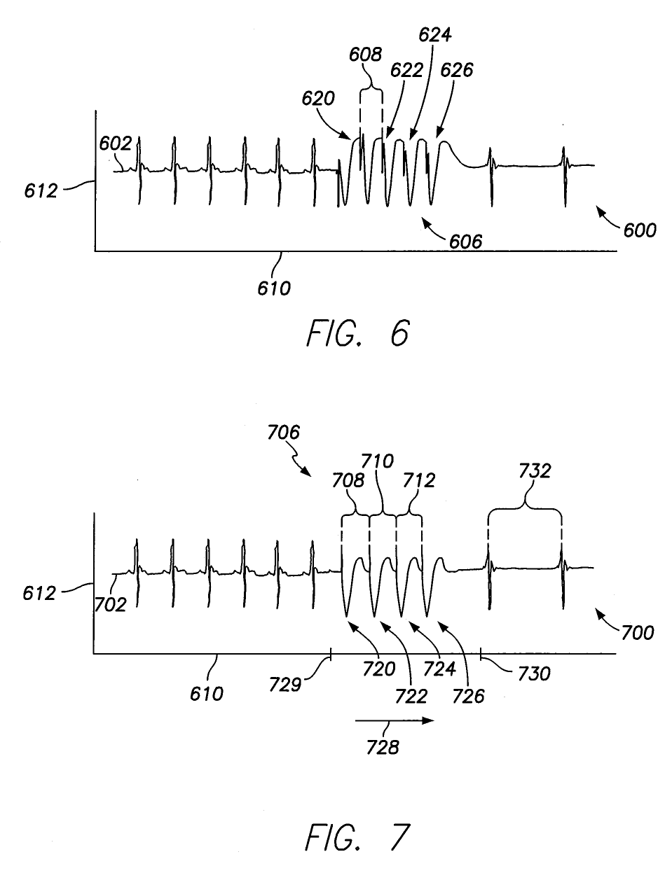

FIG. 6 is a graphical representation 600 of a cardiac electrical signal 602 sensed by the LPM 104 with a predetermined number of ATP bursts 606. The horizontal axis 610 represents time and a vertical axis 612 represents current and/or voltage. The ATP therapy is based on a predetermined number of ATP bursts 606 formed from ATP pulses 620-626. It should be noted, that although a single ATP burst 606 is shown in FIG. 6 in other embodiments the ATP therapy may include more than one ATP burst 606. Each ATP burst 606 of the ATP therapy may be separated by a number of heart beats. The ATP pulses 620-626 are shown as bi-phasic pulses, however in other embodiments the ATP pulses 620-626 may be or include one or more mono-phasic pulses, tri-phasic pulses, or the like. Each ATP pulse 620-626 is delivered by the LPM 104 at a fixed pulse interval 608. For example, the LPM 104 delivers each ATP pulse 622 after the preceding ATP pulse 620 after a fixed pulse interval 608.

Optionally, the ATP pulse intervals (e.g., the pulse intervals 708-712) may decrease or increase within the ATP burst 706. FIG. 7 is a graphical representation 700 of a cardiac electrical signal 702 sensed by the LPM 104 with a predetermined number of ATP bursts 706 formed from ATP pulses 720-726. The ATP pulses 720-726 are separated by pulse intervals 708-712. Each pulse interval 708-712 has a different length in time, such that, the subsequent pulse interval 708-712 in the direction of an arrow 728 is shorter than the previous pulse interval 708-712. For example, the pulse interval 710 is shorter than the pulse interval 708. In another example, the pulse interval 712 is shorter than the pulse interval 710. It should be noted that although the ATP pulses 720-726 are shown as bi-phasic pulses, in other embodiments the ATP pulses 720-726 may be or include one or more mono-phasic pulses, tri-phasic pulses, or the like.

Optionally, the LPM 104 may configure the ATP bursts 606 and 706 based on an ATP limit, such that the ATP therapy delivered by the LPM 104 is below the predetermined ICD threshold, to not trigger the S-ICD 106 to deliver ICD shock therapy. The predetermined ICD threshold may be based on two characteristics of the cardiac FOI. For example, the predetermined ICD threshold may be set at 220 beats per minute for a twelve interval period, which may correspond to twelve consecutive R-R intervals of approximately 273 milliseconds. Based on the predetermined ICD threshold, an ATP burst with twelve consecutive pulses separated by a pulse interval of 273 milliseconds may trigger the S-ICD 106 to deliver the ICD shock therapy.

In at least one embodiment, the ATP limit may correspond to one of the characteristics of the predetermined ICD threshold. For example, the ATP limit may correspond to a minimum pulse interval based on the 220 beats per minute of the predetermined ICD threshold. The LPM 104 may have the ATP limit set at or below 273 milliseconds such that the pulse intervals (e.g., the fixed pulse interval 608, the pulse intervals 708-712) are greater than or equal to the ATP limit. It should be noted, that although the pulse intervals are limited, each ATP burst may include more than twelve ATP pulses.

Additionally or alternatively, the ATP limit may correspond to a number of ATP pulses (e.g., ATP pulses 620-626, ATP pulses 720-726) within the ATP burst (e.g., ATP burst 606, ATP burst 706) based on the twelve interval period of the predetermined ICD threshold. For example, the LPM 104 may have the ATP limit set below twelve pulses, such that the ATP bursts delivered by the LPM 104 have less than twelve pulses. It should be noted, that although the number of ATP pulses within the ATP burst is limited, the pulse intervals within the ATP burst may be at or less than 273 milliseconds. In at least one embodiment, the ATP limit may correspond to both a number of ATP pulses and a minimum pulse interval

At 312, the method 300 determines if the cardiac FOI still satisfies an arrhythmia criteria after the predetermined number of ATP bursts are delivered. For example, the controller circuit 212 may compare the cardiac FOI, from the cardiac electrical signal 602, 702 after the ATP therapy with the arrhythmia threshold 510.

If the cardiac FOI, after the ATP therapy still satisfies the arrhythmia criteria, then at 318, the method 300 generates a series of arrhythmia emulating (AE) pulses 820-828 from at least one electrode 206-208 of the LPM 104. The series of AE pulses 820-828 may be configured to increase or decrease the appearance and/or occurrence of the cardiac FOI by mimicking or substituting portions of the cardiac electrical signal 806 generated by the heart 108. The series of AE pulses 820-828 overlaid or combined with the cardiac electrical signal 806 simulate an electrophysiologic pattern of at least one of the VF and/or VT that may be generated by the heart 108. For example, the AE pulses 820-828 generated by the LPM 104 may adjust the cardiac FOI over the predetermined ICD shock threshold. The series of the AE pulses 820-828 may be configured to have a frequency within a bandwidth of a cardiac event sensing channel (e.g., VF, VT) of an implantable cardioverter device, such as the S-ICD 106. For example, the frequency content of the AE pulses 820 may have components within a bandwidth range of 0-10 kHz based on the bandwidth of the cardiac event sensing channel of the S-ICD 106. It should be noted that although the AE pulses 820-828 are shown as bi-phasic bursts, in other embodiments the AE pulses 820-828 may be or include one or more mono-phasic bursts, tri-phasic bursts, or the like.

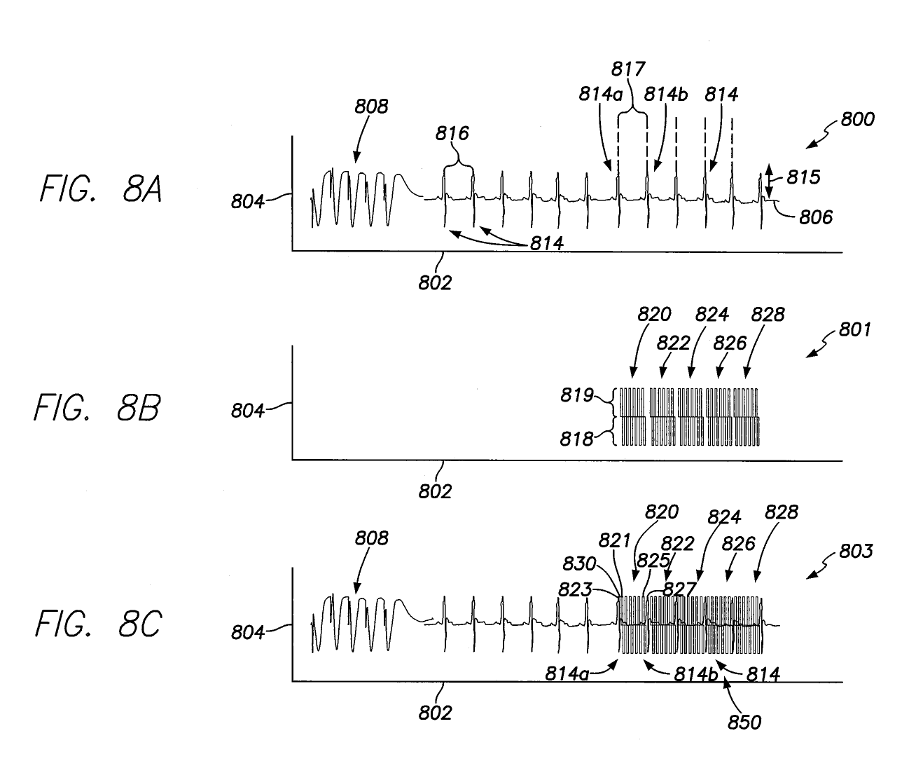

FIG. 8A is a graphical representation 800 of the cardiac electrical signal 806 sensed by the LPM 104. The horizontal axes 802 represents time and the vertical axes 804 may represent current or voltage (e.g., electrical potential). The LPM 104, as described above, may determine the ATP-responsive change in the cardiac FOI (e.g., heart rate) after the LPM 104 delivers an ATP therapy 808. The LPM 104 may determine the heart rate by measuring an R-R interval 816 between the QRS complexes 814.

For example, the LPM 104 may determine that the heart rate after the ATP therapy 808 is within the VT zone 514 at 170 beats per minute based on the R-R interval 816 of approximately 352 milliseconds. It should be noted, that in at least one embodiment the LPM 104 may determine the heart rate based on an average of R-R intervals over a series of QRS complexes 814 of the cardiac electrical signal 806. The LPM 104 may generate the series of AE pulses 820-828 between the QRS complexes 814 to reduce the R-R interval 816 simulating a heart rate (e.g., the cardiac FOI) to be above the predetermined ICD shock threshold.

FIG. 8B is a graphical representation 801 of the AE pulses 820-828 generated by the LPM 104 and delivered by at least one of the electrodes 206-208. FIG. 8C is a graphical representation 803 of the series of AE pulses 820-828 overlaid with the cardiac electrical signal 806 to form a simulated electrophysiologic pattern 850. Each of the series of AE pulses 820-828 are shown between two QRS complexes 814. For example, the series of AE pulses 820 are shown positioned between the QRS complexes 814a-b. The series of AE pulses 820 subdivides an R-R interval 817 between the QRS complexes 814a-b to create an adjusted R-R interval 830. An amplitude 818 and 819 of the AE pulses may be based on an R amplitude 815 of the cardiac electrical signal 806. The adjusted R-R interval 830 is configured to trigger the S-ICD 106 to deliver ICD shock therapy.

For example, when the cardiac FOI is above the predetermined ICD threshold the S-ICD 106 delivers ICD shock therapy. The predetermined ICD threshold may be set at 220 beats per minute for a twelve interval period, which may correspond to twelve consecutive R-R intervals of approximately 273 millisecond. The LPM 104 subdivides the 352 millisecond R-R interval 817 with the series of AE pulses 820. The frequency of the series of AE pulses 820 may be set by the controller circuit 212 at 20 Hertz corresponding to one AE pulse approximately every 50 milliseconds or six AE pulses within the R-R interval 817. The adjusted R-R interval 830, corresponds to the amount of time to a subsequent AE pulse (e.g., R-wave peak 823 to the AE pulse 821) and/or R-wave peak (e.g., AE pulse 825 to the R-wave peak 827) between the QRS complexes 814a-b, which is approximately 50 milliseconds (simulating 1200 beats per minute). Based on the adjusted R-R interval 830, the simulated electrophysiologic pattern 850 is over 220 beats per minute over a twelve interval period (e.g., AE pulses 820-828).

In another example, the frequency of the AE pulses 820 may be set by the controller circuit 212 at 120 Hertz, corresponding to one AE pulse approximately every 8.3 milliseconds or approximately forty-two AE pulses within the R-R interval 817. The adjusted R-R interval 830 is approximately 8.3 milliseconds (simulating 7228 beat per minute). It should be noted that in other embodiments the AE pulses 820-828 may have frequencies and/or a bandwidth of 20-120 Hertz.

Optionally, if the cardiac FOI, after the ATP therapy no longer satisfies the arrhythmia criteria and/or when the series of AE pulses 820-828 are generated at 318, the method 300 may adjust the predetermined number of ATP bursts 606, 706. For example, the controller circuit 212 may reduce the number of the predetermined number of ATP bursts 606, 706 if the ATP-responsive change in the cardiac FOI resulted in the cardiac FOI to not satisfy the arrhythmia criteria. In another example, the controller circuit 212 may increase the number of the predetermined number of ATP bursts 606, 706 if the ATP-responsive change in the cardiac FOI resulted in the cardiac FOI to continue to satisfy the arrhythmia criteria at 312. Optionally, the controller circuit 212 may change the length of the ATP pulse intervals (e.g., the fixed ATP pulse interval 608, the pulse intervals 708-712) based on the ATP-responsive change in the cardiac FOI. Additionally or alternatively, the controller circuit 212 may change the type of ATP therapy delivered by the LPM 104. Optionally, the controller circuit 212 may change the predetermined number of ATP bursts 606, 706 based on the number of electrical shocks detected by the shock detector circuit 210 (e.g., at 322) over a period of time (e.g., between clinical visits, last programming of the LPM 104). In at least one embodiment, the controller circuit 212 may change the predetermined number of ATP bursts 606, 706 based on a number of cycles or times the LPM 104 has delivered ATP therapy over a period of time.

At 320, the method 300 administers ICD shocking therapy from at least one electrode of an ICD (e.g., the S-ICD 106). For example, the cardiac event sensing channel of the S-ICD 106 may sense the simulated electrophysiologic pattern 850, which includes the cardiac electrical signal 806 of the heart 108 overlaid with the AE pulses 820-828 delivered by the LPM 104 through at least one of the lead electrodes 110-114. Based on the simulated electrophysiologic pattern 850, the S-ICD 106 may determine a cardiac FOI, such as heart rate based on measuring the adjusted R-R interval 830. The S-ICD 106 may determine that the heart rate is 1200 beats per minute based on the adjusted R-R interval 830 of 50 milliseconds. The S-ICD 106 compares the cardiac FOI with the predetermined ICD shock threshold, for example, at 220 beat per minute and may deliver the ICD shock therapy through at least one of the lead electrodes 110-114 if the measured cardiac FOI is over the ICD shock threshold.

At 322, the method 300 detects an electrical shock from a shock detector (e.g. the shock detector circuit 210) of the LPM 104 corresponding to the ICD shock therapy.

At 324, the method 300 delivers post-shock pacing from at least one of the electrodes 206-208 of the LPM 104. For example, after the LPM 104 detects the electrical shock from the shock detector circuit 210, the LPM 104 may deliver post-shock pacing, such as post-shock bradycardia pacing, according to a WI mode from at least one of the electrodes 206-208.

In at least one embodiment, during the post-shock pacing the LPM 104 may measure the cardiac FOI from the cardiac electrical signal of the heart 108 to determine whether the cardiac FOI is within the VT zone 514. If the cardiac FOI is within the VT zone 514, the LPM 104 may deliver AE pulses to simulate electrophysiologic pattern that includes the cardiac FOI above the predetermined ICD shock threshold. Optionally, the VF threshold 512 may also correspond to the predetermined ICD shock threshold. The series of AE pulses may be detected by an implantable cardioverter device (e.g., the S-ICD 106) positioned remote from the LPM 104 proximate to the heart 108.

FIG. 9 is a schematic block diagram of at least one embodiment of the S-ICD 106 and shows the functional elements of the S-ICD 106 enclosed in the housing 120. A plurality of terminals or Hermetic feedthroughs 929-931 may conduct electrode signals through the housing 120 into the lead 128 and interface with the lead electrodes 110-114 of the S-ICD 106. Additionally or alternatively, one feedthrough 929-931 may conduct electrode signals for a plurality of the lead electrodes 110-114. The housing 120 contains a battery 914 to supply power for pacing, sensing, and/or other functions of the S-ICD 106 described herein. The housing 120 also contains sensing circuitry 902 that may include a sensing amplifier 932 and a supra-ventricular tachycardia (SVT) discriminator 916. It should be noted that in other embodiments, the sensing circuit 902 may further employ one or more low power, precision amplifiers with programmable gain and/or automatic gain control, bandpass filtering, and threshold detection circuit to selectively sense the cardiac signal of interest. The sensing circuitry is configured to sense cardiac activity through a cardiac event sensing channel of the S-ICD 106 through one or more of the lead electrodes 110-114. In the example of FIG. 2, a single sensing circuit 902 is illustrated. Optionally, the S-ICD 106 may include multiple sensing circuits, similar to the sensing circuit 902, where each sensing circuit is coupled to one or more electrodes and controlled by the controller circuit 912 to sense electrical activity detected at the corresponding one or more electrodes 110-114. The sensing circuit 902 may operate in a unipolar sensing configuration or in a bipolar sensing configuration.

The sensing amplifier 932 is selectively coupled to one or more of the lead electrodes 110-114 through the switch 926. The switch 926 determines the sensing polarity of the cardiac signal by selectively closing the appropriate switches based on a control signal 928 from the controller circuit 912. The output of the sensing circuitry 932 is connected to the controller circuit 912 which, in turn, triggers or inhibits the shocking circuit 910 in response to the absence or presence of cardiac activity. The sensing amplifier 932 receives a control signal 946 from the controller circuit 912 for purposes of controlling the gain, threshold, polarization charge removal circuitry (not shown), and/or the timing of any blocking circuitry (not shown) coupled to the inputs of the sensing amplifier 932. For example, the sensing amplifier 932 may have a low pass frequency (e.g., 10-120 Hertz) is adjusted by the controller circuit 912. The low pass frequency may correspond to the bandwidth of the cardiac event sensing channel of the S-ICD 106.

Additionally or alternatively, the output of the sensing amplifier 932 may be supplied to the SVT discriminator 916. The SVT discriminator 916 is configured to output a detection signal 924, based on the sensed cardiac activity, to the controller circuit 912 if an SVT is detected within the heart 108. In at least one embodiment, the controller circuit 912 may not deliver the ICD shock therapy based on the detection signal 924. For example, the controller circuit 912 may have determined that the cardiac FOI is above the predetermined ICD shock threshold indicating a possible VT. If the controller circuit 912 receives the detection signal 924 indicating SVT rather than the VT, the controller circuit 912 may not instruct the shock circuit 910 to deliver the ICD shock therapy to one or more of the lead electrodes 110-114.

In at least one embodiment, the LPM 104 may adjust when to generate the series of AE pulses based on the presence or absence of the ICD shock therapy. The delivering of the ATP bursts 606, 706 may be dependent on whether a detection signal from the SVT discriminator 916 is detected by the LPM 104. For example, the LPM 104 may generate the series of AE pulses 820-828 after determining the cardiac FOI of the cardiac electrical signal is within the VT zone 514 forming the simulated electrophysiologic pattern 850. The cardiac event sensing channel of the S-ICD 106 senses the simulated electrophysiologic pattern 850, and determines the adjusted cardiac FOI (e.g., based on the adjusted R-R interval 830) is above the predetermined ICD shock threshold. The SVT discriminator 916 may detect an SVT from the electrophysiologic pattern 850 and output the detection signal 924 to the controller circuit 912 withholding the ICD shock therapy. Based on the absence of the ICD shock therapy, after delivery of the series of AE pulses 820-828, the LPM 104 may determine that the cardiac electrical signal corresponded to an SVT event. The LPM 104 may log characteristics of the cardiac electrical signal (e.g., shape, amplitude, frequency, or the like) in memory 220, which may be compared to other sensed cardiac electrical signals by the controller circuit 212. If the cardiac electrical signal includes the logged characteristics, the controller circuit 212 may determine the sensed cardiac electrical signals correspond to an SVT event and not deliver the AE pulses 820-828

The SVT discriminator 916 may detect SVT events based on sensed cardiac activity from one (e.g., local chamber of the LPM 104) or more chambers (e.g., local chamber and adjacent chamber) of the heart 108 corresponding to the position of the lead electrodes 110-114 sensed by the sensing amplifier 932. For example, for a single chamber the SVT discriminator 916 may compare an area of difference between sensed QRS complexes of the sensed cardiac activity that is a part of the cardiac FOI to a template QRS complex (e.g., based from a sinus rhythm of the heart 108) stored in memory 920. If the morphology of the sensed QRS complex (e.g., area of difference between the sensed QRS complex and the template QRS complex) is below an SVT threshold, the SVT discriminator 916 may output the detection signal 924. In another example, the SVT discriminator 916 may receive sensed cardiac activity from multiple chambers of the heart 108, such as the right atrium and right ventricle, from the sensing amplifier 932. The SVT discriminator 916 may compare cardiac FOIs from sensed cardiac activity for each chamber to determine whether the cardiac activity corresponds to an SVT.

Optionally, the sensing circuitry 902 may include a pulse sensing amplifier (not shown) configured to receive communication pulses from the LPM 104 emitted by one or more of the electrodes 206-208. The pulse sensing amplifier may include higher frequencies than the bandwidth of the cardiac event sensing channel of the S-ICD 106. For example, the pulse sensing amplifier may have a bandwidth from 10 Hertz to 100 Kilohertz. Optionally, the bandwidth of the pulse sensing amplifier may be adjusted by the controller circuit 912. The communication pulses may be transmitted during the absolute refractory period (e.g., after the R-wave) of the heart 108 based on the sensed cardiac electrical signal. The amplitude of the communication pulses may be sub-threshold or supra-threshold pulses relative to the threshold potential of the heart 108 that triggers an action potential.

Optionally, the communication pulses may be delivered by the LPM 104 concurrently with the AE pulses, the ATP therapy, and/or post-shock therapy. For example, the LPM 104 may deliver communication pulses and chopped ATP bursts simultaneously or concurrently from one or more stimulation electrodes 206-208. The chopped ATP burst may represent a subdivided ATP burst having fewer ATP pulses, relative to the ATP burst, with preceding and/or subsequent communication pulses that may alternate between each ATP pulse of the chopped ATP burst. Optionally, the communication pulses may have a pulse width between 2 and 1500 microseconds. It should be noted, in other embodiments the pulse width may be less than or greater than 2 and/or 1500 microseconds, respectively.

One or more of the lead electrodes 110-114 receive stimulus high voltage pulse(s), conforming to the ICD shock therapy, generated from the shocking circuit 910 for delivery by one or more of the lead electrodes 110-114 coupled thereto. The shocking circuit 910 is controlled by the controller circuit 912 via a control signal 982. The shocking circuit 910 may generate shocking pulses of low (e.g., up to 0.5 joules), moderate (e.g., 0.5-10 joules), or high energy (e.g., 10 to 40 joules), as controlled by the controller circuit 912.

The controller circuit 912 is illustrated as including timing control circuitry 927 to control the timing of the ICD shock therapy (e.g., atrio-ventricular (AV) delay etc.). The timing control circuitry 927 may also be used for the timing of refractory periods, blanking intervals, noise detection windows, evoked response windows, alert intervals, marker channel timing, and so on. The controller circuit 912 also has an arrhythmia detector 922 which uses the predetermined ICD shock threshold for detecting arrhythmia conditions, for example, based on one or more cardiac FOI measured by the sensing circuitry. Although not shown, the controller circuit 912 may further include other dedicated circuitry and/or firmware/software components that assist in monitoring various conditions of the heart 108 and managing pacing therapies.

The S-ICD 106 may further include an analog-to-digital (A/D) data acquisition system (DAS) 950 coupled to one or more electrodes 110-114 via the switch 926 to sample cardiac signals across any pair of desired electrodes. The data acquisition system 950 may be configured to acquire intracardiac electrogram signals, convert the raw analog data into digital data, and store the digital data for later processing and/or telemetric transmission to an external device 954 (e.g., a programmer, local transceiver, or a diagnostic system analyzer). The data acquisition system 950 is controlled by a control signal 956 from the controller circuit 912.

The controller circuit 912 is coupled to the memory 920 by a suitable data/address bus 962. The programmable operating parameters used by the controller circuit 912 may be stored in memory 920 and used to customize the operation of the S-ICD 106 to suit the needs of a particular patient. Such operating parameters define, for example, pulse duration of the ICD shock therapy, electrode polarity, rate, sensitivity, automatic features, arrhythmia detection criteria (e.g., the predetermined ICD shock threshold), the pulse amplitude, wave shape and vector of each shocking pulse to be delivered to the heart 108.

The operating parameters of the S-ICD 106 may be non-invasively programmed into the memory 920 through a telemetry circuit 964 in telemetric communication via a communication link 966 with the external device 954. The telemetry circuit 964 allows intracardiac electrograms and status information relating to the operation of the S-ICD 106 (as contained in the controller circuit 912 and/or the memory 920) to be sent to the external device 954 through the established communication link 966.

The battery 914 provides operating power to all of the components in the S-ICD 106. The battery 914 is capable of operating at low current drains for long periods of time, and is capable of providing high-current pulses. The battery 914 also desirably has a predictable discharge characteristic so that elective replacement time can be detected. As one example, the S-ICD 106 employs lithium/silver vanadium oxide batteries.

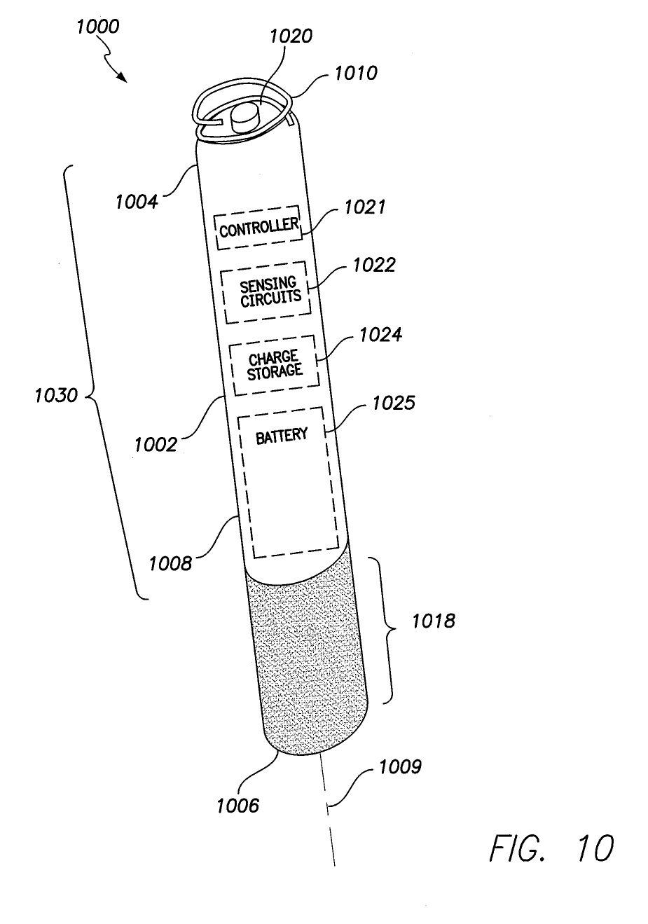

FIGS. 10 illustrate an LPM 1000 in more detail. The LPM 1000 comprises a housing 1002 having a distal base 1004, a distal top end 1006, and an intermediate shell 1008 extending between the distal base 1004 and the distal top end 1006. The shell 1008 is elongated and tubular in shape and extends along a longitudinal axis 1009. The LPM 1000 includes a battery 1025 for power supply.

The base 1004 includes one or more electrodes, such as an inner electrode 1020, which is securely affixed thereto and projected outward. For example, the outer element 1010 may be formed as large semi-circular spikes or large gauge wires that wrap only partially about the inner electrode 1020. The element 1010 is wound around electrode 1020. The element 1010 may be used for affixing the LPM 1000 to the tissue. In this case, the element 1010 may be inactive electrically and may be coated with an insulator like parylene or may be simply not connected to the case or any associated circuitry. Alternatively, the element 1010 may also be used as an electrode to pick up the local potentials from the tissue surrounding the electrode 1020 and the element 1010. This allows for exclusive detection of electrograms from the local tissue (in the local chamber of the LPM 1000).

Included at the distal top end 1006 of the LPM 1000 is an electrode 1018. Electrode 1018 is electrically connected to the sensing circuits 1022 and is used to perform pulse sensing. The pulse sensing is performed between the inner electrode 1020 and the electrode 1018. In between the electrode 1020 and the electrode 1018 is an insulated region 1030 that separates the electrodes 1020 and 1018. The region 1030 may be insulated with a parylene coating.

Pulses are generated by the charge storage circuit 1024 and are emitted between the electrode 1020 (e.g., configured as a cathode) and the electrode 1018 (e.g., configured as an anode). Because of the relatively large separation between the electrodes 1018 and 1020, a dipole field generated in the tissue by the electrode 1010 and 1020 may facilitate communication to another device (e.g., the LPM 104, the S-ICD 106). So the relatively large separation between the electrodes 1020 and 1018 facilitates transmission of the information carried on the communication pulses over relative large distances in the body. For example, the distance between electrodes 1020 and 1018 may be one-half to two-thirds of the overall length of the LPM 1000 (e.g., over 10 mm, 5-20 mm, up to 30 mm). If the element 1010 is electrically active, it may also be used for sensing pulses using the sensing circuits 1022.

The LPM 1000 may include a charge storage unit 1024 and sensing circuit 1022 (e.g., sensing circuitry 232) within the housing 1002. The sensing circuit 1022 senses intrinsic activity, while the charge storage unit 1024 stores high or low energy amounts to be delivered in one or more stimulus pulses. The sensing circuit 1022 senses intrinsic and paced events. The electrode 1020 and/or element 1010 (e.g., configured as an electrode) may be used to deliver lower energy or high energy stimulus, such as pacing pulses, AE pulses, ATP bursts, cardioverter pulse trains, or the like. The electrodes 1020, 1018 may also be used to sense electrical activity, such as physiologic and pathologic behavior and events and provide sensed signals to the sensing circuit 1022. The electrodes 1020, 1018 are configured to be joined to an energy source, such as a charge storage unit 1024. The electrodes 1020, 1018 receive stimulus pulse(s) from the charge storage unit 1024. The electrodes 1020, 1018 may be configured to deliver high or low energy stimulus pulses to the myocardium.

The LPM 1000 includes a controller 1021, within the housing 1002 to cause the charge storage unit 1024 to deliver activation pulses through each of the electrodes 1020, 1018 in a synchronous manner, based on information from the sensing circuit 1022, such that activation pulses delivered from the inner electrode 1020 are timed to initiate activation in the adjacent chamber. The controller 1021 performs the various operations described herein in connection alternative embodiments for the systems and the methods. The stimulus pulses are delivered synchronously to local and distal activation sites in the local and distal conduction networks such that stimulus pulses delivered at the distal activation site are timed to cause contraction of the adjacent chamber in a predetermined relation to contraction of the local chamber.

The controller circuits 212, 912, and the controller 1021 may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the controller circuits 212, 912, and the controller 1021 may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The controller circuits 212, 912, and the controller 1021 may execute a set of instructions that are stored in one or more storage elements, in order to process data. The storage elements may also store data or other information as desired or needed. The storage element may be in the form of an information source or a physical memory element within the controller circuits 212, 912, and the controller 1021. The set of instructions may include various commands that instruct the controller circuits 212, 912, and the controller 1021 to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f), unless and until such claim limitations expressly use the phrase “means for” followed by a statement of function void of further structure.

What is claimed is:

1. A method for managing tiered tachycardia therapy, the method includes:
measuring a cardiac feature of interest (FOI) from a cardiac electrical signal of a heart sensed from at least one electrode of a leadless cardiac pacemaker (LPM);
detecting when the cardiac FOI satisfies an arrhythmia criteria;
delivering anti-tachycardia pacing (ATP) therapy to the heart using the at least one electrode of the LPM when the cardiac FOI satisfies the arrhythmia criteria; and
delivering a series of arrhythmia emulating (AE) pulses configured to emulate a cardiac arrhythmia to the heart using the at least one electrode of the LPM if the cardiac FOI in cardiac electrical signals measured subsequent to the delivered ATP therapy satisfy the arrhythmia criteria.

2. The method of claim 1, wherein the series of AE pulses is configured to simulate an electrophysiologic pattern for at least one of ventricular fibrillation or ventricular tachycardia.

3. The method of claim 1, further comprising detecting the series of AE pulses at an implantable cardioverter device (ICD) positioned remote from the LPM proximate to the heart.

4. The method of claim 1, wherein the series of AE pulses are configured to have a frequency within a bandwidth of a cardiac event sensing channel of an implantable cardioverter device (ICD).

5. The method of claim 1, wherein the series of AE pulses have a frequency and the bandwidth of 20-120 Hz.

6. The method of claim 1, wherein the arrhythmia criteria represents a ventricular tachycardia (VT) zone and the cardiac FOI represents a heart rate or a predetermined time period at that heart rate, such that the ATP therapy is delivered when the heart rate or the heart rate for the predetermined time period is within the VT zone.

7. The method of claim 1, further comprising detecting an electrical shock from a shock detector circuit of the LPM corresponding to an implantable cardioverter defibrillator (ICD) shock therapy; and
delivering post-shock pacing from the plurality of electrodes of the LPM based on the output of the shock detector circuit.

8. The method of claim 1, further comprising adjusting the ATP therapy when the series of AE pulses are generated by the LPM.

9. The method of claim 1, wherein delivering the ATP therapy is dependent on whether a detection signal from a supra-ventricular tachycardia (SVT) discriminator is detected by the LPM, the SVT discriminator configured to output the detection signal if an SVT is detected within the heart.

10. The method of claim 1, wherein a first electrode of the LPM is used for the measuring operation, a second electrode of the LPM is used for the delivering operation, and a third electrode of the LPM is used for the generating of the series of AE pulses operation.

11. A system for managing tiered tachycardia therapy comprising:
a leadless pacemaker (LPM), wherein the LPM includes sensing circuitry within a housing configured to sense a cardiac electrical signal of a heart through at least one electrode; and
a controller circuit within the housing of the LPM, the controller circuit configured to:
measure a cardiac feature of interest (FOI) from the cardiac electrical signal;
detect when the cardiac FOI satisfies an arrhythmia criteria;
deliver anti-tachycardia pacing (ATP) therapy to the heart using the at least one electrode of the LPM when the cardiac FOI satisfies the arrhythmia criteria;
generate a series of arrhythmia emulating (AE) pulses configured to emulate a cardiac arrhythmia if the cardiac FOI of cardiac signals received subsequent to the delivery of ATP therapy satisfy the arrhythmia criteria; and
deliver the series of AE pulses to the heart using the at least one electrode of the LPM.

12. The system of claim 11, wherein the series of AE pulses is configured to simulate an electrophysiologic pattern for at least one of the ventricular fibrillation or ventricular tachycardia.

13. The system of claim 11, further comprising an implantable cardioverter device (ICD) having a first sensing circuitry configured to detect the series of AE pulses through at least one lead electrodes, the ICD is positioned remote from the LPM proximate to the heart.

14. The system of claim 13, wherein the series of AE pulses are configured to have a frequency within a bandwidth of a cardiac event sensing channel of the first sensing circuitry.

15. The system of claim 11, wherein the series of AE pulses have a frequency and the bandwidth of 20-120 Hz.

16. The system of claim 11, wherein the arrhythmia criteria represents a ventricular tachycardia (VT) zone and the cardiac FOI represents a heart rate or a predetermined time period at that heart rate, such that the controller circuit is further configured to deliver the ATP therapy when the heart rate or the heart rate for the predetermined time period is within the VT zone.

17. The system of claim 11, wherein the LPM includes a shock detector circuit configured to generate a detector signal corresponding to an implantable cardioverter defibrillator (ICD) shock therapy; and
the controller circuit is further configured to deliver post-shock pacing from at least one of the electrodes of the LPM based on the output of the shock detector circuit.

18. The system of claim 11, wherein the controller circuit is further configured to adjust the predetermined number of ATP bursts based when the series of AE pulses are generated by the LPM.

19. The system of claim 11, wherein the delivering operation by the controller circuit of ATP bursts is dependent on whether a detection signal from a supra-ventricular tachycardia (SVT) discriminator is detected by the LPM, the SVT discriminator configured to output the detection signal if an SVT is detected within the heart.

20. The system of claim 1, wherein a first electrode of the LPM is used for the measuring operation by the controller circuit, a second electrode of the LPM is used for the delivering operation by the controller circuit, and a third electrode of the LPM is used for the generating of the series of AE pulses operation by the controller circuit.
